(19) United States
(12) Patent Application Publication
(10) Pub. No.: US 2004/0014637 A1
(43) Pub. Date: Jan. 22, 2004

(54) Mechanism for the inactivation of insulin receptor and uses in the treatment of type II diabetes

(75) Inventors: Barry I. Posner, Westmount (CA); John J.M. Bergeron, Pointe-Claire (CA)

Correspondence Address: OGILVY RENAULT, Suite 1600, 1981 McGill College Ave., Montreal, QC, H3A 2Y3, CA

(73) Assignee: McGill University

(21) Appl. No.: 10/367,821

(22) Filed: Feb. 19, 2003

Related U.S. Application Data
(63) Continuation-in-part of application No. 09/312,393, filed on May 14, 1999, now abandoned.

(30) Foreign Application Priority Data
May 15, 1998 (CA) 2,237,791

(51) Int. Cl. A61K 39/395; A61K 31/00; G01N 33/53

(52) U.S. Cl. 514/1; 435/7.1; 424/146.1

(57) ABSTRACT

The present invention relates to a method for the treatment of Type II Diabetes Mellitus patients, which comprises administering an acidotropic agent to the patient in an amount sufficient for neutralizing intraendosomal pH or preventing lowering of intraendosomal pH; thereby activating insulin receptor kinase of the patient and preventing insulin resistance. The present invention also relates to a method for the screening of an acidotropic agent suitable for the treatment of Type II Diabetes Mellitus.

### RELATED APPLICATIONS

This application is a Continuation-In-Part of application Ser. No. 09/312,393 filed on May 14, 1999, which is still pending, and which is claiming the benefit of priority of Canadian patent application serial number 2,237,791 filed on May 15, 1998. All of the above-noted applications are hereby incorporated by reference.

### BACKGROUND OF THE INVENTION

(a) Field of the Invention

The invention relates to the mechanism for the inactivation of insulin receptor (IR) and its uses in the treatment of type II diabetes.

(b) Description of Prior Art

The insulin receptor is a heterotetrameric glycoprotein composed of 2 extracellular α-subunits bearing insulin binding sites, and 2 transmembrane β-subunits possessing tyrosine kinase activity in their cytosolic domains. Following insulin binding to the insulin receptor kinase (IRK) in intact cells, there is rapid internalization of insulin-IRK complexes into endosomes (ENs) (the endosomes thus include insulin receptors) and equally rapid activation of the IRK, as manifested by increased autophosphorylation on β-subunit tyrosine residues and augmented kinase activity toward exogenous substrates. Unlike other receptor tyrosine kinases, the IRK does not appear to recruit signaling proteins directly, but phosphorylates adaptor proteins,. including insulin receptor substrate-1 (IRS-1), insulin receptor substrate-2 (IRS-2) and SHC which function as docking entities to entrain the insulin signaling sequence. The observation that the activated IRK is internalized to ENs is consistent with the occurrence of transmembrane signaling intracellularly (Bevan, A. P. et al. (1996) Trends Endocrinol. Metab. 7, 13-21). Indeed studies in liver parenchyma have shown that the accumulation of activated IRKs exclusively in ENs is sufficient to promote IRS-1 tyrosine phosphorylation (Bevan, A. P. et al. (1995) J. Biol. Chem. 270, 10784-10791). In adipocytes it has also been shown that internal membranes are the principal sites where IRS-1 phosphorylation and P13 kinase activation occur.

Given the above considerations the mechanisms controlling receptor function in ENs are clearly important for understanding the regulation of insulin signaling. Studies have shown that the level of IRK tyrosine phosphorylation and hence activity is altered and ultimately reduced by an IRK-associated phosphotyrosine phosphatase in ENs (Faure, R. et al. (1992) J. Biol. Chem. 267, 11215-11221). Discovery of the endosomal acidic insulinase (EAI) (Authier, F. et al. (1994) J. Biol. Chem. 269, 3010-3016) has demonstrated a mechanism by which intraendosomal insulin concentration may be reduced hence decreasing the proportion of IRKs occupied by ligand. The observation that endosomal insulin degradation was ATP dependent led to the demonstration in a cell free system that endosomal acidification both promotes insulin dissociation from the IRK and subsequent degradation of free insulin by the EAI (Doherty, J. J. et al. (1990) J. Cell Biol. 110, 35-42).

It would be highly desirable to be provided with the mechanism for the inactivation of insulin receptor and its uses in the treatment of type II diabetes.

### SUMMARY OF THE INVENTION

One aim of the present invention is to provide the mechanism for the inactivation of insulin receptor and its uses in the treatment of type IT diabetes.

In accordance with the present invention there is provided a method for the treatment of Type II Diabetes Mellitus patients, which comprises administering an acidotropic agent to the patient in an amount sufficient for neutralizing intraendosomal pH or preventing lowering of intraendosomal pH; thereby activating insulin receptor kinase of the patient and preventing insulin resistance.

The acidotropic agent may be chloroquine.

In accordance with the present invention there is also provided a method for the screening of an acidotropic agent suitable for the treatment of Type II Diabetes Mellitus, which comprises the steps of:

a) incubating endosomes with a screened acidotropic agent and ATP;

b) measuring insulin receptor kinase (IRK) activity, wherein an active insulin receptor kinase is indicative of a suitable acidotropic agent.

The measuring of IRK activity may be effected using an anti-IRK antibody.

The antibody may be α960 and ¹²⁵I-GAR.

In accordance with the present invention there is provided the use of an ATP-driven proton pump to serve as a target for the design of acidotropic agents for the treatment of Type II Diabetes Mellitus.

### BRIEF DESCRIPTION OF THE DRAWINGS

FIG. 1 illustrates the effect on IRK activity of incubating intact ENs containing insulin receptor with ATP;

FIG. 2 illustrates the absence of inhibitory effect of nonhydrolysable ATP analogues on IRK activity;

FIG. 3A illustrates the effect on IRK activity of incubating PM and ENs containing insulin receptor with ATP;

FIG. 3B illustrates the effect on EGF Receptor Autophosphorylation of incubating ENs containing EGF receptor with ATP;

FIG. 4 illustrates the effect of the ATPase inhibitor, Bafilomycin, on the ATP-dependent deactivation of endosomal IRK activity;

FIG. 5 illustrates the phosphoamino acid analyses of the IRK β-subunit after incubating intact ENs containing IR with 1 and 5 MM γ-³²P ATP;

FIG. 6 illustrates the effect of inhibiting PTP activity by bpV(phen) on ATP-dependent changes in endosomal IRK activity;

FIG. 7 illustrates the inhibition by ATP of the kinase activity of endosomal IRK is independent of the state of β-subunit tyrosine phosphorylation; and

FIG. 8 illustrates the effect of ATP on DTT-dependent reduction of endosomal IRK species.

### DETAILED DESCRIPTION OF THE INVENTION

In the present study we report that preincubation of isolated ENs containing IR with adenosine 5′triphosphate (ATP), under conditions promoting acidification and maximal insulin dissociation, resulted in decreased insulin binding capacity of the IRK. Of perhaps greater importance is our observation of marked acidification-dependent deactivation of IRK activity towards exogenous substrates. Our data indicate that this latter phenomenon derives from an acidification dependent conformational change in the intraluminal aspect of the endosomal IRK with attendant deactivation of the cytosolic tyrosine kinase. These data thus identify other processes leading to attenuation of the activated state of the IRK, and hence transmembrane signaling during the course of endocytosis.

Following internalization insulin undergoes dissociation from its receptor and selective degradation within endosomes (ENs) in an ATP-dependent manner [Doherty, J. J. et al, J. Cell Biol. (1990) 110:35-42]. We now observe that incubating ENs containing IR with ATP markedly decreased binding activity for ¹²⁵I-insulin but not ¹²⁵I-hGH in a temperature dependent manner. This effect was not observed with ADP, AMP, adenosine, pyrophosphate and non-hydrolysable analogs of ATP showing that the γ-phosphate high-energy bond was necessary. As the ATP concentration was increased from 0.1 to 1 mM there was a corresponding increase in β-subunit tyrosine phosphorylation and exogenous kinase activity of the insulin receptor kinase (IRK) assayed after solubilization and partial purification on Wheat Germ Agglutinin (WGA)-column chromatography. However, incubations at higher ATP concentrations resulted in a dramatic decrease in β-subunit tyrosine phosphorylation and IRK activity. Since this was not observed with non-hydrolysable analogs of ATP nor when plasma membrane (PM) was incubated under the same conditions it was considered to be dependent on ATP hydrolysis and to be compartment specific. Furthermore no ATP-dependent inhibition of EGF receptor kinase autophosphorylation was observed, indicating relative specificity for the IRK. The inhibition of IRK tyrosine phosphorylation and activity, on incubating ENs containing IR with 5 mM ATP, was completely reversed by Bafilomycin A1 showing that activation of endosomal proton pump(s) was involved in this process. Examination of events on the cytosolic face of ENs containing IR demonstrated that the inhibition of IRK was not due to serine/threonine phosphorylation or PTP activation. Thus 2-D phosphoamino acid analysis of the IRK did not reveal an augmentation of ³²P-phosphoserine/threonine content of the IRK on incubating at 5 mM ATP. Nor was the ATP inhibitory effect influenced by in vitro or in vivo inhibition of PTPs using bpV(phen). Prior phosphorylation of the β-subunit with 1 mM ATP did not prevent the inhibition of IRK activity on incubating with 5 mM ATP. This dissociation between IRK activity and β-subunit tyrosine phosphorylation suggested that the IRK had undergone a conformational change leading to an inactive form of the kinase. To evaluate this latter possibility we assessed the effect of DTT to reduce the heterotetrameric to dimeric form of the IRK. The α₂β₂ was the predominant form of IRK detected following incubations in the absence of DTT, whereas the αβ dimer was the predominant form seen in the presence of DTT. In contrast, incubating ENs containing IR with DTT in the presence of 5 mM ATP yielded α₂β₂ as the predominant form detected. Thus, ATP-dependent endosomal acidification promotes the termination of transmembrane signaling by sustaining the dissociation-degradation sequence for intraendosomal insulin, and by effecting a conformational change of the IRK leading to the attenuation of its activity.

Materials and Methods

Animals

Female Sprague-Dawley rats (140-160 g body weight) were purchased from Charles River Ltd. (St. Constant, Quebec) and were fasted overnight prior to killing.

Reagents

Porcine insulin (26.8 IU/mg) was a gift from Eli Lilly Research Laboratories (Indianapolis Ind.). Human growth hormone (hGH, 2.2 IU/mg was from the NIH pituitary hormone and antisera program (Baltimore, Md.). Carrier- free [¹²⁵I]-iodine and [γ-³²P] ATP (1000-3000 Ci/mmol) were purchased from New England Nuclear Dupont (Wilmington, Del.). NaCl, MgSO₄, trichloroacetic acid and glycerol were from Anachaemia Ltd. (Lachine, Quebec) Wheat Germ Agglutinin-Sepharose™ 6-MB (WGA) and protein-A Sepharose™ were from Pharmacia fine chemicals (Dorval, Quebec). Nucleosides tri-, di-, and monophosphates, AMP-P-C-P, AMP-P-N-P, and AMP-P-S-P were from Boehringer Mannheim (Laval, Quebec). Chemicals for SDS-PAGE were from Bio-Rad Laboratories. Kodak™ X-Omat, AR films were purchased from Picker International Canada (Montreal, Quebec). Immobilon was from Millipore Canada Ltd. (Mississauga, ON). Bafilomycin A1, Poly Glu:Tyr (4:1), N-acetyl-D-glucosamine and other chemicals were from Sigma (St. Louis, Mo.). The peroxovanadium compound bpV(phen) was synthesized and purified as per Bevan et al. (Bevan et al. (1995) J. Biol. Chem. 270, 10784-10791).

Antibodies

Antibody to the juxtamembrane domain (residues 942-968) of the insulin receptor (α960) and to phosphotyrosine (αPY) were prepared and purified by chromatography on Protein-A Sepharose™ and phosphotyrosine affi-gel columns respectively (Bevan et al. (1995) J. Biol. Chem. 270, 10784-10791). Affinity purified goat anti-rabbit antibodies (whole molecule) were purchased from Sigma and iodinated to a specific activity of 6×10⁸ dpm/μg IgG using a chloramine T procedure (¹²⁵I-GAR) (Posner, B. I. et al. (1982) J. Biol. Chem. 257, 5789-5799).

Subcellular Fractions, Binding and Protein Determination

Rats were anaesthetized with ether and were injected via jugular vein with a dose (per 100 g body weight) of insulin (1.5 μg), or bpV(phen) (0.6 μmol), dissolved in 0.2 ml. phosphate buffered saline (pH 7.4) −0.1% BSA. Animals were killed by decapitation at 2 and 15 mins postinjection of insulin and bpV(phen) respectively (Faure, R. et al. (1992) J. Biol. Chem. 267, 11215-11221). Livers were rapidly excised, placed in ice-cold homogenizing buffer (50 mM HEPES (pH 7.4), 0.25 M sucrose, 1 mM PMSF, 1 MM MgCl₂, 1 mM benzamidine) and minced before homogenization. Combined ENs and PM fractions were prepared (Faure et al. (1992) J. Biol. Chem. 267, 11215-11221) except that the buffer used throughout was that in which the livers were minced (see above). These subcellular fractions have been characterized in detail both morphologically and biochemically. Hormone binding was assayed with ¹²⁵I-insulin or ¹²⁵I-hGH prepared to a specific activity of 100-200 μCi/μg using the chloramine-T method (Posner, B. I. et al. (1982) J. Biol. Chem. 257, 5789-5799). Protein content in the fractions was determined by a modification of Bradford's method using serum albumin as a standard (Khan, M. N. et al. (1985) Diabetes 34, 1025-1030).

Insulin Receptor Phosphotyrosine Content

The phosphotyrosine content of endosomal IRs (ENs containing IR) was determined by subjecting lectin column (WGA-Sepharose™)-purified preparations to SDS-PAGE and immunoblotting with α960 and αPY (Faure et al. (1992) J. Biol. Chem. 267, 11215-11221).

Insulin Receptor Kinase Assays

Insulin receptors from the subcellular fractions were partially purified by lectin column chromatography using WGA-Sepharose™ 6MB columns (Bevan et al. (1995) J. Biol. Chem. 270, 10784-10791). Receptor content in each preparation was estimated by assessing specific binding of ¹²⁵I-insulin after overnight incubation at 4° C. (Bevan et al. (1995) J. Biol. Chem. 270, 10784-10791). To measure the IRK activity of the purified insulin receptors to phosphorylate an exogenous substrate, aliquots of receptor (generally 20 μl) containing 10-15 fmoles of insulin binding were added to a reaction mixture containing 87.5 mM HEPES (pH 7.4), 40 mM MgCl₂, 25 μM [γ-³²P]-ATP (5 μCi/assay tube) and 5 mg/ml of poly Glu:Tyr (4:1) in a final volume of 100 μl. After 10 min at room temperature, the reactions was terminated by spotting 50 μl aliquots on Whatman 3 MM filters. After air drying the filters were immersed in a solution of ice-cold 10% TCA-10 mM Na pyrophosphate prior to washing and counting (Bevan et al. (1995) J. Biol. Chem. 270, 10784-10791) to determine the amount of IRK catalyzed transfer of ³²P from ATP to poly Glu:Tyr (4:1).

Insulin Receptor Autophosphorylation and Phosphoamino Acid Analyses

ENs containing IR were removed from the 0.6/1.0 M sucrose interface of the gradient used in endosomal purification, and diluted with 0.25 M sucrose to a final protein concentration of 50 μg/ml. A 30 ml aliquot was centrifuged at 200,000×gₐᵥ for 40 mins prior to resuspending in 500 μl of cell-free system buffer (44 mM HEPES (pH 7.4), 0.55 M sucrose, 333 mM KCl, 11 mM NaCl, 11 mM MgCl₂). Incubations were initiated by adding 500 μl of 10 mM Tricine buffer containing either 2 or 10 mM ATP at a specific activity of 0.5 mCi ³²P/mmol. After incubating for 15 min at 37° C. the reaction was stopped by adding an equal volume of ice-cold 20 mM HEPES, 0.25 M sucrose, 2 mM PMSF, 2 mM benzamidine, 4 mM Na orthovanadate and 80 mM Na fluoride. To measure the IRK autophoshorylation and/or phosphoamino acid activity of IRK, ENs containing IR were solubilized by incubating for an additional 30 mins at 4° C. in a final concentration of 1% Triton X-100, 20 mM pepstatin, 20 mM leupeptin, and 10 mg/ml aprotinin, after which IRs were selectively purified from the ENs by immunoprecipitation with α960, washed in buffer, subjected to SDS-PAGE, and transferred to Immobilon™-P membranes. The membranes were then subjected to autoradiography and/or amino acid analyses (Faure et al. (1992) J. Biol. Chem. 267, 11215-11221)to determine the amount of ³²P transferred from ATP.

Insulin Receptor Under Nonreducing Conditions

Endosomal pellets containing IR were resuspended (500 μg protein/ml) in a final concentration of 50 mM Tris (pH 6.9), 10% glycerol and 20 mM N-ethylmaleimide. Samples were incubated at room temperature for 5 mins and then solubilized without heating in 2.3% SDS-0.05% bromophenol blue before subjecting to SDS-PAGE in the absence of reductants (Faure et al. (1192) J. Biol. Chem. 267, 11215-11221).

EGF Receptor Autophosphorylation in the Presence and Absence of ATP

EGF (10 μg/100 g body weight) was injected via the jugular vein into anesthetized rats. The animals were sacrificed by decapitation 15 mins later and ENs containing EGF receptor were prepared as noted above. Resuspended ENs containing EGF receptor were incubated for 15 mins with 5 mM ATP after which auto-phosphorylation was initiated by adding ³²P-ATP. The reaction was stopped after 15 mins of incubation at 37° C. by adding Laemmli sample buffer after which aliquots (100 μg protein) were subjected to SDS-PAGE, alkali digestion, and radioautography as described previously (Faure, R. et al. (1992) J. Biol. Chem. 267, 11215-11221).

Results

Following its in vivo administration ¹²⁵I-insulin concentrates in ENs containing IR with maximum levels attained at 2 mins postinjection. When subsequently incubated in vitro intraendosomal ¹²⁵I-insulin undergoes degradation in an ATP-dependent manner due to the activity of an endosomal acidic insulinase (EAI) (Authier, F. et al. (1994) J. Biol. Chem. 269, 3010-3016; Doherty, J. J. et al. (1990) J. Cell Biol. 110, 35-42). Previous work has shown that both ¹²⁵I-insulin degradation and dissociation from its receptor are both temperature and ATP-dependent processes. In earlier studies we showed that ATP induces acidification of the intraendosomal space and that this promotes both ligand dissociation from its receptor and the activity of EAI (Authier, F. et al. (1994) J. Biol. Chem. 269, 3010-3016; Doherty, J. J. et al. (1990) J. Cell Biol. 110, 35-42).

Effect of ATP on Insulin Binding and IRK Activity

In the present study we wished to assess whether other processes might be involved in reducing the association of intraendosomal insulin with its receptor. We examined the effect of incubating intact ENs containing IR with increasing concentrations of ATP on subsequently measured insulin and hGH binding in both solubilized and WGA-purified receptors. Table 1 illustrates that as the ATP concentration was increased (0.1, 1, 3, 5, and 10 mM) there was a progressive decrease of insulin but not hGH binding.

TABLE 1
The effect of preincubating ENs containing IR with
increasing ATP concentrations on ¹²⁵I-insu1in and ¹²⁵I-
hGH binding

| | Solubilized endosomes | | WGA- purified endosomes | |
|---|---|---|---|---|
| ATP (MM) | Ins | HGH | Ins | hGH |
| 0 | 100 | 100 | 100 | 100 |
| 0.1 | 103.6 ± 2.5 | 105.0 ± 5.1 | 107.7 ± 1.5 | 88.7 ± 3.4 |
| | (6) | (3) | (6) | (3) |
| 1 | 90.9 ± 4.2 | 84.4 ± 6.6 | 123.9 ± 6.2 | 93.3 ± 5.1 |
| | (6) | (3) | (6) | (3) |
| 3 | 96.7 ± 2.6 | ND | 99.3 ± 0.9 | ND |
| | (3) | | (3) | |
| 5 | 84.2 ± 2.7 | ND | 52.1 ± 1.2 | ND |
| | (3) | | (3) | |
| 10 | 26.5 ± | 98.3 ± 3.5 | 8.4 ± 0.2 | 75.2 ± 3.5 |
| | 21.7^{a} | (3) | (6) | (6) |
| | (13) | | | |
| ^{a}p < 0.001 Control versus 10 mM ATP | | | | |
| ENs containing IR from insulin-injected rats were preincubated with ATP at 37° C. for 15 min, solubilized and assayed for insulin or hGH binding before and after WGA-purification as described in Materials and Methods. Specific binding was expressed as a percent of that in control ENs containing IR preincubated without ATP. Binding in control ENs containing IR was: Solubilized ENs (per 50 μg protein) - insulin, 16.6 ± 3.1% (N = 13); hGH, 15.2 ± 1.7% (N = 3); | | | | |
| #WGA-purified (per 1 μg protein) - insulin, 17.4 ± 4.9% (N = 6); hGH, 11.4 ± 1.0 (N = 3). Values are the means ± SD. ND, not determined. | | | | |

As observed in Table 2 the decrease of insulin binding was not produced by ADP, AMP, Na pyrophosphate and adenosine. Nor did nonhydrolyzable ATP analogues mimic the effect of ATP.

TABLE 2
The effect of different nucleotides on endosomal ¹²⁵I-
insulin binding activity
²⁵I-Insulin specific binding (%)

| | | | WGA- |
|---|---|---|---|
| | | Solubilized | purified |
| Addition | | endosomes | endosomes |
| None | 0 | 100 | 100 |
| ATP | 0.1 mM | 103.4 ± 2.5 | 108.1 ± 2.0 |
| | 1 mM | 95.5 ± 14.2 | 92.2 ± 2.2 |
| | 10 mM | 43.6 ± 6.3 | 25.2 ± 5.2 |
| | (4° C.) | 112.0 ± 3.3 | — |
| | 10 mM | | |
| ADP | 10 mM | 96.3 ± 6.4 | 82.7 ± 5.2 |
| AMP | 10 mM | 98.2 ± 2.5 | 98.9 ± 1.9 |
| Adenosine | 10 mM | 94.5 ± 4.1 | 105.9 ± 3.3 |
| NaPiPi | 10 mM | 87.4 ± 3.1 | 97.8 ± 2.3 |
| AMP—P—C—P | 10 mM | 109.1 ± 0.9 | 95.1 ± 2.0 |
| AMP—P—N—P | 10 mM | 112.2 ± 2.8 | 99.1 ± 1.0 |
| AMP | 10 mM | 104.1 ± 2.8 | 103.7 ± 2.8 |

Results are the mean 2 to 5 experiments ± SD. Endosomes containing IR from insulin-injected rats were preincubated with the noted concentration of nucleotides at 37° C. for 15 min, solubilized and assayed for insulin binding before and after WGA-purification as described in Experimental Procedures. The specific binding observed was normalized to those levels obtained with endosomes preincubated without nucleotide. All values are the means ± SD.

In addition the loss of binding activity was temperature dependent as no changes were observed when ENs containing IR were incubated on ice in the presence of 10 mM ATP (Table 2). These data strongly suggest that the ATP-dependent effect was specific for insulin and necessitated hydrolysis of the γ-phosphate of ATP.

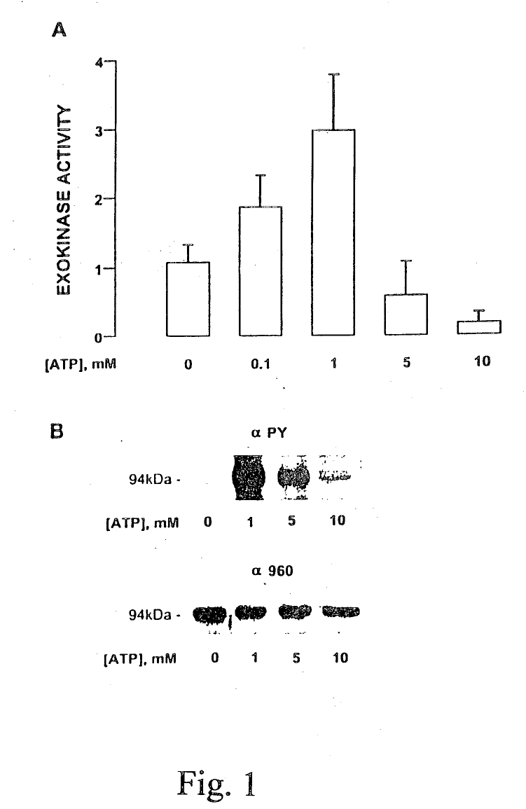

On preincubating ENs containing IR with increasing concentrations of ATP we also observed an effect on IRK activity and phosphotyrosine content (FIG. 1). Rats were injected with insulin (1.5 μg/100 g body weight) and sacrificed after 2 min. Hepatic EN fractions containing IR were prepared and immediately incubated for 15 min at 37° C. with ATP at the indicated concentration. The ENs were then solubilized and IRK was purified from the ENs by lectin column (WGA-Sepharose™) chromatography. FIG. 1A illustrates the effect of ATP on IRK activity. Equal amounts of WGA-purified IRK was assayed for exogenous tyrosine kinase activity using Poly (Glu:Tyr), (4:1) as substrate. Exokinase activity was expressed as pmol/10 min/10 fmol insulin binding. Each point reflects the mean±S.E. of determination from 4-6 separate experiments.

In the absence of ATP, IRK activity reflected the effect of preinjected insulin as demonstrated on previous occasions. At 0.1 and 1.0 mM ATP we observed an increase whereas at 5 mM ATP there was a marked decrease in IRK activity even to a level below that observed in the absence of ATP. At 10 mM ATP, IRK activity was virtually abolished. IRK phosphotyrosine content was markedly increased on incubating ENs containing IR with 1 mM ATP (FIG. 1, Panel B). FIG. 1B illustrates the effect of ATP on β subunit phosphotyrosine content. WGA eluates (5 μg protein) were subjected to SDS-PAGE in 7.5% gels followed by transfer of proteins to Immobilon-P membranes. Membranes were probed with αPY or α960 antibodies followed by incubation with ¹²⁵I-GAR as second antibody and exposed for autoradiography. At 5 and 10 mM ATP this level was significantly reduced and virtually abolished respectively compared to 1 mM ATP while the amount of β-subunit remained unchanged.

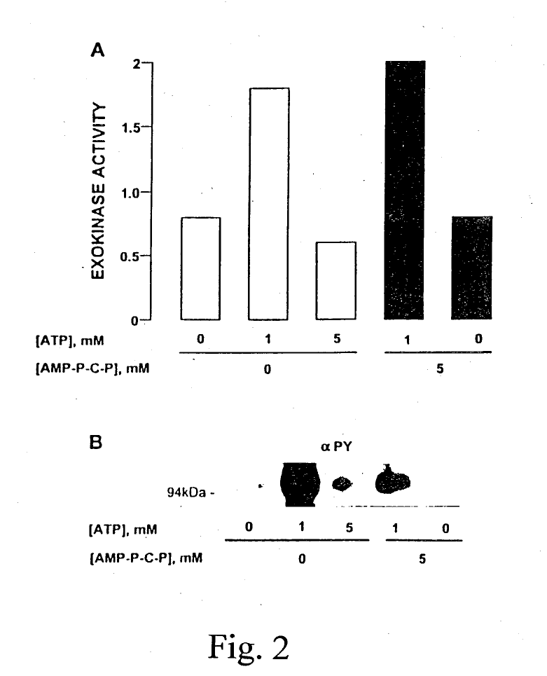

To verify the importance of the high energy bond of ATP in producing the above changes in IRK activity and tyrosine phosphorylation, we incubated ENs containing IR with 0 and 1 mM ATP in the presence (5 mM) or absence of the non-hydrolysable ATP analog, AMP-P-C-P. The results of FIG. 2 show that incubating ENs containing IR with 5 mM AMP-P-C-P had no effect on the level of IRK activity observed in the presence or absence of 1 mM ATP. Two mins. following insulin injection (1.5 μg/100 g body weight) rats were sacrificed and hepatic EN fractions containing IR were prepared and immediately incubated for 15 min at 37° C. with ATP in the absence or in presence of 5 mM AMP-P-C-P. The ENs were solubilized and IRK purified by lectin column (WGA-Sepharose™) chromatography. FIG. 2 (Panel A) illustrates the effect of AMP-P-C-P on IRK activity. WGA-purified IRK was assayed for exogenous tyrosine kinase activity using Poly (Glu:Tyr) (4:1) as substrate. Exokinase activity was expressed as pmol/10 min/10 fmol insulin binding. FIG. 2 (Panel B)illustrates the effect of AMP-P-C-P on β subunit phosphotyrosine content. WGA eluates (5 μg protein) were subjected to SDS-PAGE in 7.5% gels. Proteins were transferred to Immobilon-P membranes which were probed with αPY antibody followed by incubation with ¹²⁵I-GAR and exposed for autoradiography.

The effect of the analog to reduce IRK phosphotyrosine concentration to a modest extent may reflect some inhibition of IRK autophosphorylation.

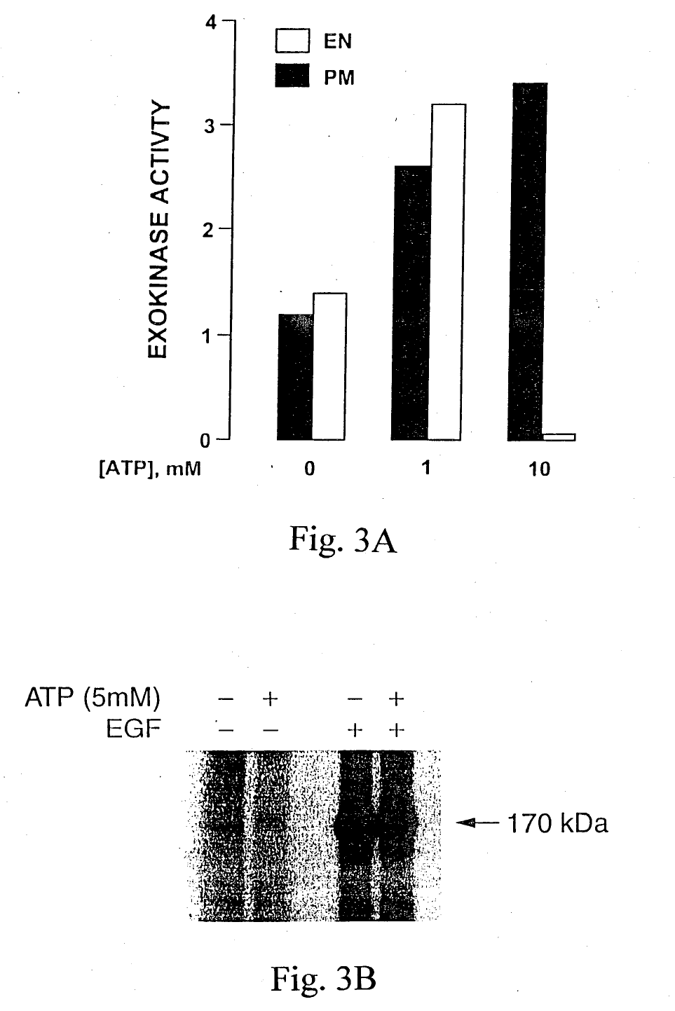

We next evaluated whether the observed ATP-dependent attenuation of the IRK activation state is specific to the endosomal compartment. On preincubating PM containing IR with ATP we observed an augmentation of activity of WGA-purified IRK (FIG. 3A). Hepatic PM and EN fractions containing IR, prepared at 30 secs and 2 mins after injecting rats with insulin (1.5 μg /100 g body weight), were immediately incubated for 15 min at 37° C. with ATP at the indicated concentrations. Cell fractions were solubilized and IRK, purified by lectin column (WGA-Sepharose™ ) chromatography, was assayed for exogenous tyrosine kinase activity using Poly (Glu:Tyr) (4:1) as substrate. Exokinase activity was expressed as pmol/10 min/10 fmol insulin binding.

At 10 mM the increase in PM IRK activity was greater than that observed at 1 mM in sharp contrast to what was found in ENs containing IR where preincubation with 10 mM ATP suppressed IRK activity completely.

We then determined whether preincubation with ATP attenuated EGF receptor autophosphorylation. On incubating ENs containing EGF receptor from EGF-treated rats with 5 mM ATP no significant attenuation of ³²P-labeling of the EGF receptor was observed (FIG. 3B). EN fractions, prepared 15 mins after injecting rats with EGF (10 μg/100 g body weight), were incubated in the presence or absence of ATP (5 mM) prior to conducting autophosphorylation with ³²P-ATP, SDS-PAGE, and autoradiography.

Role of Endosomal Acidification

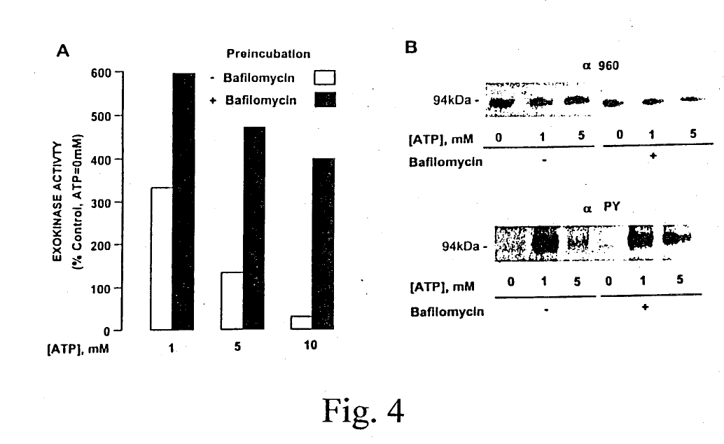

Various studies have established that in ENs there is a progressive luminal acidification through the action of ATP-dependent proton pumps. To evaluate the role of ATP-dependent acidification on the IRK activation state we incubated ENs containing IR with Bafilomycin A1, a potent inhibitor of endosomal ATPases, in the presence and absence of ATP. Hepatic ENs containing IR were prepared 2 min. after insulin injection (1.5 μg /100 g body weight), and were preincubated in the absence or presence of 1 μM Bafilomycin A1 After 30 min at 37° C. the incubation was continued for an additional 15 min with ATP at the indicated concentrations. As noted in FIG. 4 (Panel A) coincubation with Bafilomycin produced a marked attenuation in the ability of 5 and 10 mM ATP to effect a reduction in IRK activation. FIG. 4 (Panel A) illustrates the effect of Bafilomycin A₁ on ATP-dependent inhibition of IRK activity. WGA-purified IRK tyrosine kinase activity was assayed using Poly (Glu:Tyr) (4:1) as substrate and expressed as a percent of the exokinase activity obtained from incubations conducted in the absence of ATP. IRK activity in ENs containing IR preincubation with or without Bafilomycin A₁ and before ATP additions were 1.4 and 1.3 pmol/10 min/10 fmol insulin binding respectively.

Furthermore in the presence of Bafilomycin the level of IRK tyrosine phosphorylation at 5 mM ATP was comparable to that observed at 1 mM ATP (FIG. 4, panel B). FIG. 4 (Panel B) illustrates the effect of Bafilomycin on ATP-dependent regulation of IRK β-subunit autophosphorylation. WGA eluates(5 μg protein) were subjected to SDS-PAGE in 7.5% gels followed by transfer of proteins to Immobilon-P membranes. The membranes were then probed with αPY or a960 antibodies and a ¹²⁵I-GAR as second antibody followed by autoradiography. The same finding were seen in three repeat experiments.

Studies on Attenuation of IRK-Activation

We subsequently sought to determine the mechanism by which ATP-dependent endosomal acidification results in diminution of the IRK activation state.

Serine/Threonine Phosphorylation

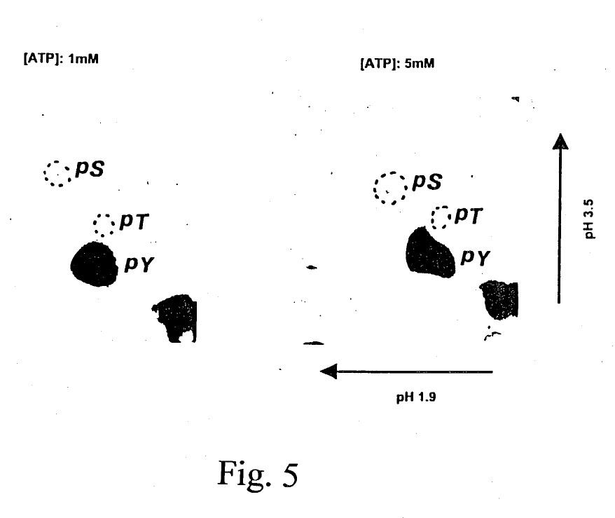

Initially we examined processes which could act on the cytosolic domain of the IRK to reduce β-subunit tyrosine phosphorylation and hence IRK activation. Previous work has shown that serine/threonine phosphorylation of the β-subunit of the IRK results in an inhibition of IRK activity. We thus determined if incubating ENs containing IR with 5 mM ATP promotes the phosphorylation of IRK on serine and threonine residues. ENs were incubated with 1 or 5 mM [γ-³²P] ATP (0.5 mCi/mmol) and IRs were purified thereafter by immunoprecipitation and SDS-PAGE and subjected to phosphoamino acid analyses. A 2 D-phosphoamino acid analysis of the β-subunit of the IRK showed no detectable ³²P phosphoserine in material from either the 1 or 5 mM ATP incubations (FIG. 5). Hepatic ENs containing IR, prepared 2 mins following insulin (1.5 μg/100 g body weight) injection, were incubated for 15 min at 37° C. with 1 or 5 mM γ-³²P ATP (0.5 mCi/mmol), solubilized and the IR purified from the ENs by immunoprecipitation with α960 antibodies. Immunoprecipitates were resolved on SDS-PAGE and transferred to Immobilon™-P membranes. Regions of the Immobilon-P membranes containing the immunoprecipitated IRK β-subunit were localized by autoradiography, excised, and subjected to acid hydrolysis to release phosphoamino acids which were separated by 2-dimensional thin layer electrophoresis (TLE). TLE was performed using equal amounts of ³²P label (500 cpm) and phosphoserine (S), phosphothreonine (T) and phosphotyrosine (Y) which were localized by ninhydrin staining. A second experiment yielded comparable results.

Activation of PTP(s)

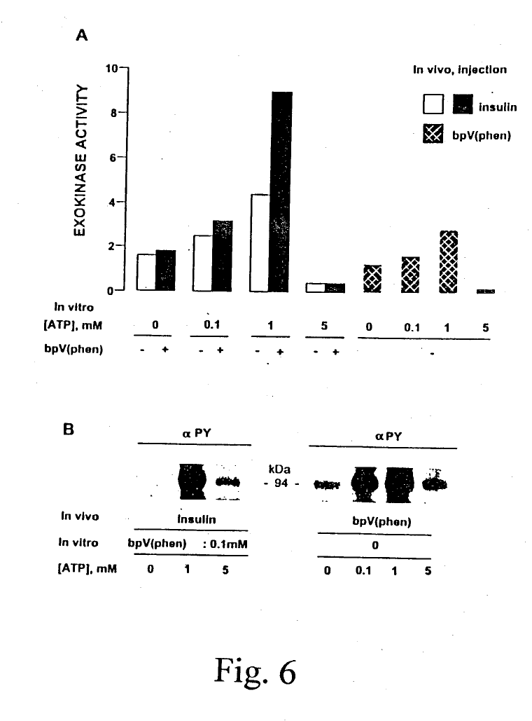

We considered the possibility that higher ATP concentrations might promote activation of endosomal PTPs and effect a reduction in the phosphotyrosine content of the β-subunit of the IRK. Such endosomal PTP activity has been previously documented (Faure, R. et al. (1992) J. Biol. Chem. 267, 11215-11221). We thus blocked PTP activity using bpV(phen), a potent PTP inhibitor. The results of FIG. 6 show that coincubating ENs containing IR with bpV(phen) did not prevent the marked suppression of IRK activity seen in the presence of 5 mM ATP. Hepatic ENs were prepared at 2 or 15 mins after insulin (1.5 μg/100 g body weight) or bpV(phen) (0.6 μmol/100 g body weight) administration respectively, and immediately incubated for 15 min. at 37° C. with ATP in the absence or presence of 0.1 mM bpV(phen). ENs containing IR were solubilized and IRK purified by lectin column (WGA-Sepharose™) chromatography. FIG. 6 (Panel A) illustrates the effect of different ATP concentrations on IRK activity. WGA-purified IRK preparations were assayed for exogenous tyrosine kinase activity using Poly (Glu:Tyr) (4:1) as substrate. Exokinase activity was expressed as pmol/10 min/10 fmol insulin binding.

Furthermore when ENs containing IR were isolated from rats pretreated with bpV(phen) the augmented IRK activity observed in this circumstance was also suppressed on incubating ENs with 5 mM ATP. Parallel findings were obtained on examining β-subunit phosphotyrosine content. Thus bpV(phen) did not influence the reduction observed in β-subunit tyrosine phosphorylation seen in the presence of 5 mM ATP (FIG. 6, panel B). FIG. 6 (Panel B) illustrates the effect of different ATP concentrations on IRK β-subunit phosphotyrosine content. WGA eluates (5 μg protein) were subjected to SDS-PAGE in 7.5% gels, transferred to Immobilon-P membranes which were probed with αPY and a ¹²⁵I-GAR as second antibody followed by autoradiography. Comparable results were observed in three separate studies.

We conclude that possible PTP activation plays no role in effecting a reduction of endosomal IRK function at higher ATP concentrations.

Dissociation of IRK Activity and its Autophosphorylation State

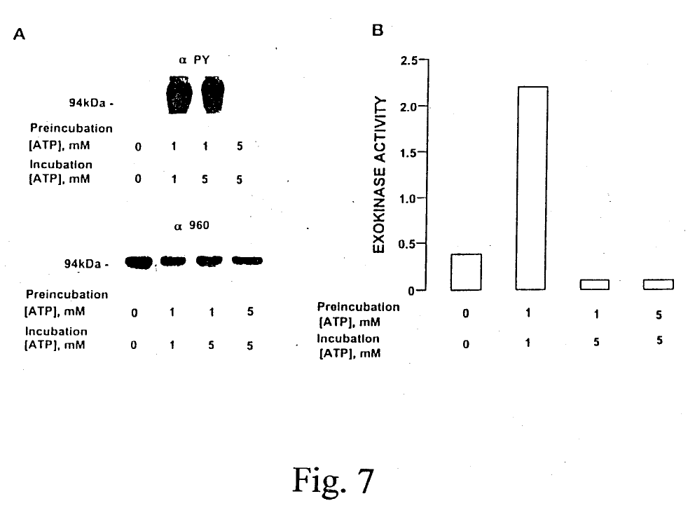

We assessed the relationship between the acidification-dependent attenuation of IRK activity and the tyrosine phosphorylation state of the β-subunit of IRK. To do this we preincubated ENs containing IR with 1 or 5 mM ATP for 15 mins followed by a second incubation with 5 mM ATP. As observed in FIG. 7 preincubation with 1 mM ATP followed by a second incubation with 5 mM ATP resulted in marked reduction of IRK activity in the presence of a substantial retention of its phosphotyrosine content. Hepatic ENs containing IR were prepared 2 mins after insulin injection (1.5 μg/100 g body weight) and were preincubated for 15 min at 37° C. with 0, 1 or 5 mM ATP. This was followed by a second incubation 15 min with ATP at the indicated concentrations. The ENs were solubilized and IRK purified by lectin column (WGA-Sepharose™) chromatography. FIG. 7 (Panel A) illustrates the phosphotyrosine content of the β subunit of the IR. WGA eluates (5 μg protein) were subjected to SDS-PAGE in 7.5% gels, and proteins were transferred to Immobilon-P membranes which were subsequently probed with αPY or α960 antibodies and ¹²⁵I-GAR as second antibody prior to autoradiography. FIG. 7 (Panel B) illustrates the IRK activity. WGA-purified IRK activity was assayed, using Poly (Glu:Tyr) (4:1) as substrate, and expressed as pmol/10 min/10 fmol insulin binding. Comparable results were seen in a second experiment.

Sequential incubations in 1 mM ATP had no deleterious effect on either IRK activity or phosphotyrosine content. Thus endosomal acidification results in the inactivation of an autophosphorylated IRK.

Evidence for an Acidification-Dependent Conformational Change of the Endosomal IRK

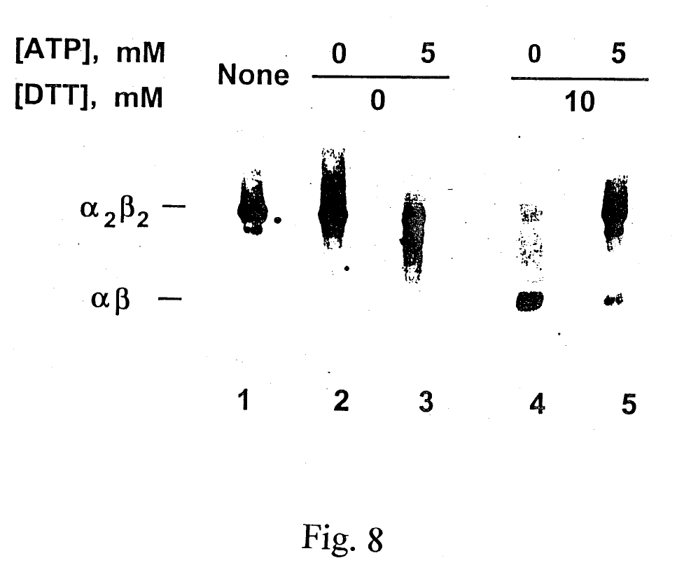

The above observation raised the possibility that intraluminal events were responsible for deactivation of the endosomal IRK. This led us to investigate whether there might be a conformational change in the IRK consequent to ATP-dependent acidification. To probe this possibility we incubated ENs containing IR with ATP in the presence or absence of 10 mM DTT to assess the ease with which Type I disulfide bonds might be reduced in the heterotetrameric molecule (α₂O₂). As seen in FIG. 8 the heterotetramer was readily identified after incubating in the presence or absence of 5 mM ATP (lanes 1 to 3). Hepatic ENs containing IR were prepared 2 mins after insulin injection (1.5 μg/100 g body weight) and were incubated for 15 min at 37° C. in the absence or presence of 10 mM DTT and 5 mM ATP. The ENs were centrifuged and the pellets resuspended and treated as described above. Samples were applied without heating to SDS-PAGE (gradient resolving gel, 3-10% acrylamide) in the absence of reductant. Proteins were transferred to Immobilon™-P membranes and probed with α960 antibodies and ¹²⁵I-GAR as second antibody prior to autoradiography. The same finding was observed in three separate experiments.

When the incubation contained 10 mM DTT the heterotetramer was readily reduced to the heterodimer (αβ) in the absence but not the presence of 5 mM ATP (lane 4 vs 5). Thus, consequent to ATP-dependent acidification of endosomes there is a change in α-subunit conformation which renders the Type I disulfide bonds relatively resistant to reduction by DTT.

Discussion

The endosomal apparatus consists of a series of distinct, non lysosomal, tubulovesicular components involved in the uptake and sorting of ligand-receptor complexes. The ENs employed in these studies were previously characterized in respect to marker enzymes, cytochemistry, electron microscopy and ligand uptake and shown to contain Golgi elements but to be substantially free of plasma membrane and other subcellular constituents (Posner, B. I. et al. (1982) J. Biol. Chem. 257, 5789-5799; Desbuquois, B. et al. (1990) Eur. J. Biochem. 193, 501-512). The addition of ATP to these ENs in a cell-free system was previously shown to augment both dissociation and degradation of internalized insulin (Doherty, J. J. et al. (1990) J. Cell Biol. 110, 35-42). This effect was due to stimulation of an ATP-dependent proton pump resulting in acidification (pH 5.5) of the intraendosomal milieu with consequent activation of a protease for which insulin serves as a relatively specific substrate (i.e. endosomal acidic insulinase, EAI) (Authier, F. et al. (1994) J. Biol. Chem. 269, 3010-3016; Doherty, J. J. et al. (1990) J. Cell Biol. 110, 35-42). Thus, by coupling dissociation at acidic pH with insulin degradation, the removal of internalized receptor-bound insulin was effected. However given the small volume of an endocytic vesicle (≅10⁻¹⁷ L), the extent of dissociation of insulin from its receptor might be expected to be limited even at pH 5.5- 6.0.

We thus postulated the existence of other mechanism(s) for abrogating the IRK activation state in ENs containing IR and in this study have identified several such processes.

Thus, incubating ENs containing IR with ATP resulted in a loss of insulin receptor binding capacity (Table 1). The effect of ATP was specific for the insulin receptor since no decrease in the hormone binding was observed for hGH (Table 1). The loss of insulin binding was not observed at 4° C., and the presence of the γ-phosphate of ATP was necessary since no effect were observed with adenosine, AMP, ADP and sodium pyrophosphate. The presence of a high energy bond was necessary as none of the non-hydrolysable analogs tested were able to reproduce the effect (Table 2). It is of interest that previous work has shown that degradation of ¹²⁵I-EGF or ¹²⁵I-prolactin was not detected in ENs (Doherty, J. J. et al. (1990) J. Cell Biol. 110, 35-42). The reduction of insulin binding activity identifies another mechanism for sustaining the dissociation-degradation sequence for insulin in ENs containing IR.

We identified another mechanism for attenuating insulin action within ENs containing IR. Whereas IRK activity increased in parallel with β-subunit phosphotyrosine content at 0.1 and 1 mM ATP, at higher ATP concentrations phosphotyrosine content and IRK activity were markedly reduced (FIG. 1). Thus an ATP dependent process is implicated in deactivation of the IRK within ENs containing IR. This was not due to proteolysis of the β-subunit since no loss of intact β-subunit was observed under these circumstances. The effect is dependent on the high energy bond of ATP, and was unique to the endosomal compartment, as in the PM IRK activity increased in parallel with the ATP concentration. ENs are known to have a slightly acidic internal milieu maintained by an ATP-dependent proton pump (Doherty, J. J. et al. (1990) J. Cell Biol. 110, 35-42). The observation that ATP-dependent inhibition of IRK activity was reversed by Bafilomycin (FIG. 4) strongly supports the idea that the intraluminal acidification of ENs, consequent to the activities of endosomal ATPases, is critical to this process. It is noteworthy that the levels of ATP promoting IRK inhibition approximate the intracellular concentrations found by several groups.

We explored the mechanism by which ATP-dependent endosomal acidification effects inactivation of the IRK. Since serine/threonine phosphorylation of the IRK has been show to reduce IRK activation, we examined the phosphoamino acid content of the endosomal IRK incubated in the presence of 1 vs 5 mM ATP. 2D-phosphoaminoacid analyses showed that the IRK did not undergo phosphorylation on serine and threonine residues at the higher ATP concentration. Thus the inhibition of the IRK activity by 5 mM ATP was not effected by serine/threonine phosphorylation of the β-subunit. The marked reduction in phosphotyrosine content of the IRK seen at 5 mM ATP was not a consequence of augmented PTP activity since bpV(phen) did not antagonize the inhibitory effect of 5 mM ATP on either IRK phosphotyrosine content or activity.

Of interest was our observation that the ATP inhibitory effect was independent of the phosphorylation state of the β-subunit (FIG. 7). This observation pointed to an intrinsic defect in IRK function and suggested that there was an alteration in structure which might account for our observations. Indeed we showed that the ability of DTT to reduce tetrameric IRK molecules was significantly decreased subsequent to the incubation of ENs containing IR with 5 mM ATP (FIG. 8). This reduced susceptibility of the Type I disulfide bond between the α and β subunits to DTT implies that there has occurred a pH-dependent structural modification of the IRK. We suggest that a conformational change of the IRK, effected by the ATP-dependent intraluminal drop in pH, was transmitted to the cytosolic domain of the IRK resulting in a decrease in IRK activity. This is indeed consistent with crystallographic studies of the IRK suggesting that, whereas activation of IRK occurs through a transautophosphorylation reaction, deactivation occurs through a cis-intramolecular mechanism (cis-inhibition). It may be that the presumed change in the intraluminal component of the IRK may be responsible for the decreased ability of the receptor to bind ligand (Table 1).

This study indicates that the regulation of the endosomal IRK activity is multifaceted and that the deactivation phase of the intracellular itinerary is made up of several discrete components. Previous work has shown that insulin signaling occurs from the endosomal system (Bevan, A. P. et al. (1996) Trends Endocrinol. Metab. 7, 13-21; Bevan, A. P. et al. (1995) J. Biol. Chem. 270, 10784-10791). The present study supports the view that there is a temporal window of signaling delimited in part by progressive acidification of ENs due to the activity of ATP-dependent proton pumping. Endosomal acidification contributes to IRK inactivation by: 1) promoting insulin dissociation from the IRK; 2) activating EAI; 3) decreasing the binding capacity of IRK; and 4) altering the conformation of the IRK so as to reduce IRK activity. Our work highlights the importance of endosomal acidification in regulating insulin receptor function.

Other studies have documented the importance of endosomal acidification in regulating a range of biological processes. Vesicular stomatitis and rabies viruses enter cells through receptor-mediated endocytosis but are rendered competent to enter cytosol only after accessing the low pH of ENs. In this environment the viral envelope undergoes a conformational transition which permits the fusion of viral membrane with membranes of the ENs. This transition involves the exposure of a hydrophobic segment within the glycoprotein whose ability to interact with membranes effects fusion and extrusion of the viral core through the wall of ENs. Low pH-driven conformational changes, occurring in ENs, have also been described for the diphtheria toxin and constitute a prerequisite for the subsequent reduction of the diphtheria toxin interchain disulfide bond, the rate-limiting step in translocation of toxin into cytosol. Recent data suggest that endosomal acidification is a key determinant in regulating the dephosphorylation and resensitization of the β-adrenergic receptor. In particular it appears that consequent to receptor internalization the association of receptor and phosphatase is facilitated in a step involving pH-sensitive conformational change(s) in receptor and/or phosphatase (Krueger, K. M. et al. (1997) J. Biol. Chem. 272, 5-8). Further studies may identify that a similar phenomenon is involved in the interaction of the IRK with endosomal PTP(s) thus constituting a factor regulating subsequent IRK dephosphorylation.

The regulation of intraendosomal pH may play a role in modulating insulin sensitivity in vivo. Thus the treatment of Type II diabetic patients with the acidotropic agent chloroquine has been shown to improve glucose metabolism (Bevan, A. P. et al. (1997). J. Biol. Chem. 272, 26833-40; Blazar, B. R. et al. (1984). Diabetes 33, 1133-7; Ilarde, A. and M. Tuck (1994). Drugs & Aging 4, 470-91; Powrie, J. K. et al. (1991). Am.J. Physiol. 260, E897-904). Since chloroquine inhibits intraendosomal insulin degradation and leads to the accumulation of intact insulin in ENs containing IR (Quatraro, A. et al. (1988). Diabete et Metabolisme 14, 666-7; Smith, G. D. et al. (1987). Br. Med. J. Clin. Res. Ed. 294, 465-7; White, N. J. et al. (1987). Lancet 1, 708-11) it has been inferred that this is the basis of its ability to improve insulin sensitivity. The present work raises the possibility that chloroquine's metabolic effects are due to an influence on IRK conformation and function. Indeed it may be that pH-dependent disturbances in IRK function contribute to the pathogenesis of Type II Diabetes Mellitus.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for screening for an acidotropic agent suitable for the treatment of Type II Diabetes Mellitus, which comprises the steps of:
a) incubating endosomes with a screened acidotropic agent and ATP, wherein said endosomes comprise insulin receptor; and
b) measuring insulin receptor kinase (IRK) activity, wherein an active insulin receptor kinase is indicative of a suitable acidotropic agent,
wherein measuring IRK activity comprises determining IRK catalyzed transfer of P from ATP to an exogenous substrate or IRK catalyzed autophosphorylation.

2. The method of claim 1, which further comprises a step i) between steps a) and b),
i) purifying said IRK from said endosomes.

3. The method of claim 2, wherein said purifying step is effected using lectin column chromatography.

4. The method of claim 3, wherein said lectin column chromatography is WGA-Sepharose™ chromatography.

5. The method of claim 2, wherein said purifying step is effected by immunoprecipitating said IRK with an anti-IRK antibody.

6. The method of claim 5, wherein said anti-IRK antibody is α960.

7. The method of claim 1, wherein a concentration of ATP of more than 1 mM is provided.

8. The method of claim 7, wherein a concentration of ATP of at least 5 mM is provided.
